(19) United States
(12) Patent Application Publication
(10) Pub. No.: US 2009/0292172 A1
(43) Pub. Date: Nov. 26, 2009

(54) Expandable Delivery Devices and Methods of Use

(75) Inventors: Bill Roskopf, Pleasanton, CA (US); Barry Weitzner, Acton, MA (US); Paul J. Smith, Smithfield, RI (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed, Inc.

(21) Appl. No.: 12/404,460

(22) Filed: Mar. 16, 2009

Related U.S. Application Data
(60) Provisional application No. 61/055,053, filed on May 21, 2008.

(51) Int. Cl. A61M 29/02; A61B 1/01

(52) U.S. Cl. 600/115; 604/96.01; 604/98.01; 604/103.11

(57) ABSTRACT

A system including an overtube configured to radially expand facilitates insertion of instruments in a patient. The system is directed to an overtube comprising an elongated body having a distal end and a proximal end, the overtube configured to radially expand from an insertion configuration to an expanded configuration, and including at least one articulation section having at least one degree of freedom and including a main channel expandable in the radial direction, and at least one additional channel. The at least one articulation section can radially expand. The system may include a radially expandable overtube having an optical channel configured to receive an optical device. The main channel and/or the at least one additional channel may be sized to receive one or more medical instruments.

### BACKGROUND

An overtube, in general, facilitates the introduction of medical instruments through a body lumen for the purpose of carrying out various medical procedures. Overtubes, therefore, can act as a guide so that another device can quickly be delivered to a point of interest, while protecting the tissue along the way. In particular, overtubes have been used in endoscopy to facilitate insertion and removal of an endoscope, to protect the esophageal lining, and to assist with removal or delivery of fluids.

### SUMMARY

Described herein are various systems and methods of using an overtube. In one aspect, a system disclosed herein includes, at least, an overtube comprising an elongate body having a distal end and a proximal end. The overtube can radially expand from an insertion configuration to an expanded configuration and can include at least one articulation section having at least one degree of freedom. A main channel within the overtube is also expandable in the radial direction. The at least one articulation section can also expand in the radial direction.

In another aspect, a system disclosed herein includes, at least, an overtube comprising an elongate body having a distal end and a proximal end, the overtube configured to radially expand from an insertion configuration to an expanded configuration, and having an optical channel configured to receive an optical device.

Further described herein are methods of inserting an expandable overtube and delivering an instrument. One exemplary method can include the step of inserting an overtube within a body, wherein the overtube is configured to expand in the radial direction, the overtube having a main channel configured to expand in the radial direction and at least one additional channel. After inserting the overtube, a user can pass at least one medical instrument through the overtube.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an illustration of an overtube in the insertion configuration in accordance with an embodiment disclosed herein;

FIG. 1B is an illustration of an expanded overtube having a channel in accordance with an embodiment disclosed herein;

FIG. 2A is a cross-sectional view of a channel divider at a mid-portion of the overtube of FIG. 1 in accordance with an embodiment disclosed herein;

FIG. 2B is a cross-sectional view of a mid-portion of the overtube of FIG. 1 in accordance with an embodiment disclosed herein;

FIG. 3A is a cross-sectional illustration of the overtube showing multiple inflation chambers and a channel in accordance with an embodiment disclosed herein;

FIG. 3B is a cross-sectional illustration of the overtube of FIG. 3A and a channel in an expanded configuration in accordance with an embodiment disclosed herein;

FIG. 4 is an illustration of an endoscopic system that may be used with an overtube in accordance with an embodiment disclosed herein;

FIG. 5 is an illustration of an articulation section that may be used with an overtube in accordance with an embodiment disclosed herein; and

FIGS. 6A-6C are illustrations of a central control shaft that may be used with an overtube in accordance with the embodiments disclosed herein.

### DETAILED DESCRIPTION

As new surgical devices and approaches are developed, applicants have found the need for new delivery devices. In particular, applicants have found that it may be desirable to increase the number and/or width of devices delivered through an access point and/or natural body lumen. Where conventional devices are delivered and/or conventional procedures are attempted through a natural orifice, traditional overtubes can be difficult to insert or are limited to standardized sizes and may potentially increase tissue trauma to the lining of a natural body lumen. Accordingly, there is room for further refinement to conventional overtubes and/or instrument delivery devices.

Disclosed herein are systems and methods for an expandable overtube. In one aspect, the system is adapted for trans-oral, trans-anal, trans-vaginal, trans-urethral, trans-nasal, trans-luminal, laparoscopic, thorascopic, orthopedic, through the ear, and/or percutaneous access. In another aspect, the described systems can be used for inspection and diagnosis in addition, or as an alternative, to surgery. Moreover, the systems described herein can perform non-medical applications, such as in the inspection and/or repair of machinery.

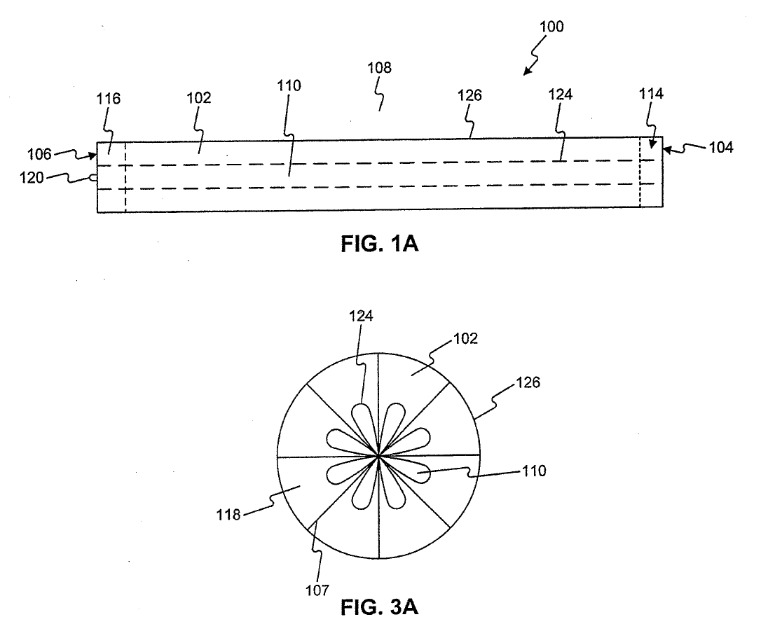

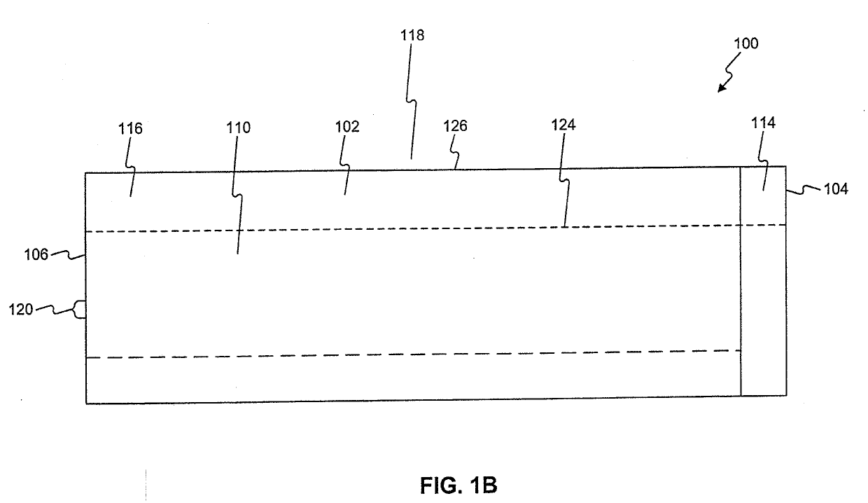

Various exemplary components of the system are described hereafter in more detail and in FIGS. 1A through 6C. Generally, a system 100 can include an elongate body that may be referred to as an “overtube” 102. Overtube 102 facilitates one time and/or repeated insertion and removal of one or more instruments, such as, for example, an endoscope, a catheter, fluid delivery tube, and/or surgical instruments from a target anatomic location.

As depicted in FIG. 1A, overtube 102 can include a distal end 104 and a proximal end 106, and a mid-portion 108 positioned therebetween. Overtube 102 can be configured for insertion through a natural orifice and/or incision of a surgical site within a patient. The outer surface of overtube 102 can include a layer of lubricous material to facilitate insertion of overtube 102 through a natural orifice or through a surgically created incision.

Overtube 102 is configured to radially expand from an insertion configuration to an expanded configuration. The insertion configuration may also approximate a shipping and/or a storage configuration. In an aspect, overtube 102 may expand from a first diameter to a second diameter. The first diameter can be less than an anatomic opening, surgical incision, percutaneous access point, body cavity, and/or body lumen. In an embodiment, the second diameter is larger than the first diameter. In a further aspect, the maximum diameter of the overtube is chosen to prevent tissue damage. In another aspect, the diameter of the overtube can correspond to an anatomic opening, surgical incision, percutaneous access point, body cavity, and/or body lumen. The size and shape of the overtube can be chosen based on the intended use and the size and shape of associated anatomical structures. In one aspect, overtubes of varying first, second, and maximum diameters can be provided as a kit.

Overtube 102 may be configured to expand in a variety of ways. In one aspect, overtube 102 is constructed with materials configured to stretch, flex, and/or deform. For instance, overtube 102 may be manufactured from at least one of elastic, polymeric, or elastomeric materials. Exemplary elastic and polymeric materials include, without limitation, polyurethane, silicone, and latex. An exemplary high strength thermoplastic elastomer is a polyether block amide (such as Pebax®). The overtube can be constructed of one or more layers of material.

In another aspect, the overtube is constructed, at least in part, of flexible, but non-stretchable and/or deformable material. Expansion can be achieved by unfolding the flexible material.

In one configuration, overtube 102 is self-collapsible, that is, overtube 102 reverts to the insertion configuration in a natural state (e.g., biased to the insertion configuration). An inflation source may be utilized to expand overtube 102 from the insertion configuration to the expanded configuration. The inflation source can be located at proximal end 106 of overtube 102, and proximal end 106 may include a port 120 for receiving the inflation source. Exemplary inflation sources include a pneumatic source for providing pressurized air or gases and a hydraulic source for providing pressurized fluids or liquids.

In another embodiment, overtube 102 is non-self-collapsible, that is, overtube 102 is biased toward, or reverts to, the expanded configuration in its natural state. An exemplary non-self-collapsible overtube 102 includes an open or closed cell foam material, the expansion of which is controlled via vacuum. Proximal end 106 of overtube 102 may comprise a port for receiving a vacuum source. In the relaxed (non-vacuum) condition, the foam is expanded, thereby expanding overtube 102. In the vacuum condition, air is withdrawn from a chamber within the overtube, and the foam collapses, thereby placing overtube 102 in the insertion configuration. After insertion, the vacuum can be released so that air or fluid may enter into the chamber and/or into the foam, and the natural resiliency of the foam will cause expansion.

In yet another embodiment, overtube 102 may be expanded by the insertion of a medical instrument therein. For example, the cross-sectional width of a medical instrument can be greater than a passageway within the overtube. Inserting the instrument can expand the overtube to accommodate the size and shape of a medical instrument. In another aspect, the expanded configuration is configured to at least partially support at least one medical instrument in translation and/or rotation.

In yet another embodiment, overtube 102 expands following exposure to biofluids and/or biomaterials, or in an aspect, the elevated temperatures of biofluids and/or biomaterials. For example, the material from which the overtube is constructed may be configured to expand following exposure to an elevated temperature relative to ambient temperature, that is, higher than about 20° C. to about 23° C. Other properties of a biofluid and/or biomaterial may also initiate expansion of the material of overtube 102. For instance, the material may be selected to expand following exposure to a pH of the biofluid, or a biofluid chemical component may initiate expansion.

Overtube 102 can be flexible (e.g., allow side to side and/or up and down bending) to permit insertion along a non-linear pathway. In addition, or alternatively, the overtube can be torqueable and/or resist longitudinal compression. For example, the materials constructing overtube can permit transmission of torque while allowing the overtube to bend.

The cross-sectional shape of the expanded overtube may be selected depending on the anatomical configuration of the targeted anatomy or the surgical approach (that is, trans-oral, laparoscopic, and the like.) In one embodiment, the cross-sectional shape of overtube 102 may be circular. In another embodiment, the cross-sectional shape may be elliptical. In yet another embodiment, the cross-sectional shape is asymmetric, for example, D-shaped.

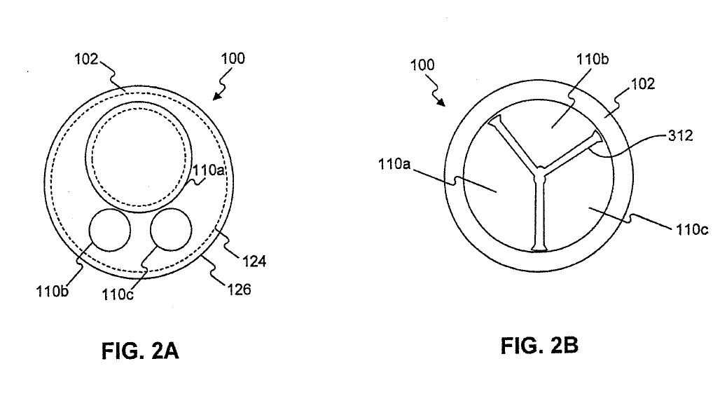

In one aspect, a wall or walls of a channel can be defined, at least in part, by an inner wall 124 of overtube 102. For example, as illustrated in FIG. 2B, a channel divider 312 can define various channels within the outer wall of the overtube. Channel divider 312 can define two, three, or more channels and can extend all or a portion of the length of overtube 102. Channel divider 312 may be constructed of materials that permit the overtube to expand.

Alternatively, or additionally, channel 110 can be fully enclosed by a channel body fixedly or detachably mated with the outer wall of overtube 102. FIG. 2A illustrates one such exemplary embodiment with channels 110b and 110c and a main channel 110a for the passage of various instruments.

In one aspect, illustrated in FIGS. 1A and 1B, a main channel may be centrally and axially positioned within overtube 102. The main channel can be sized and shaped for receiving an optical device such as, for example, an endoscope when expanded. The area adjacent to the main channel can, in one aspect, define one or more channels for the passage of instruments. In another aspect, one or more channels can be positioned along any longitudinal axis of overtube 102 (e.g., there need not be a channel extending along the central longitudinal axis of the overtube).

Each channel can receive one or more instruments, for example, an endoscope, a catheter, fluid delivery tube, surgical instruments, a guide wire or tube, and the like. In another aspect, overtube 102 can have two channels, three channels, or more than three channels. The number of channels and their particular configuration can be varied depending on the intended use of the system and the resultant number and type of surgical instruments required during a procedure. Reference may be made to a channel 110. Unless otherwise indicated, channel 110 includes embodiments directed to one or more channels or a plurality of channels. For example, overtube 102 can include a single channel adapted to receive multiple instruments or multiple channels for multiple instruments. In another exemplary embodiment, the main channel is initially sized to receive an optical device and may be additionally expanded to receive at least one other instrument.

In one embodiment, the main channel may be sized to receive a stand-alone optical device, such as an endoscope, while an additional channel may be sized to receive surgical tools, including, for example, articulating surgical tools.

In an alternative or additional aspect, optics can be integrated into overtube 102. For example, a cable running the length of overtube 102 from proximal end 106 to distal end 104 can transmit images to a viewer. The cable may be a fiber optic cable to provide overtube 102 with the ability to view and/or illuminate a body lumen as the overtube 102 is inserted into a patient. Alternatively, the cable may be an electrical cable to carry power to an image sensor and one or more light emitting diodes (LEDs) at distal end 104 of overtube 102.

A health care provider may make a diagnosis based on images the images received from an optical device. If treatment is warranted, one or more instruments may be passed through the main channel and/or additional channel to perform a treatment procedure. The main channel and/or additional channel may be expanded before the one or more instruments are inserted therein. In another embodiment, the main channel and/or additional channel are expanded as the one or more instruments are passed therethrough.

Channel 110 may be configured to radially expand from a first configuration to a second configuration. In the first configuration, channel 110 may be collapsed. In a second configuration, channel 110 may expand to a second diameter.

In one aspect, channel 110 is constructed with materials configured to stretch, flex, and/or deform. For instance, a wall of channel 110 may be manufactured from at least one of elastic, polymeric, or thermoplastic elastomeric materials. Exemplary elastic and polymeric materials include, without limitation, polyurethane, silicone, or latex. An exemplary high strength thermoplastic elastomer may be a polyether block amide (such as Pebax®). The wall can be constructed of one or more layers of material.

In one configuration, channel 110 is self-collapsible, that is, channel 110 is biased toward, or reverts to, the insertion configuration in a natural state. An inflation source can expand channel 110 and/or outer wall 126 of overtube 102. For example, inflation fluid can be delivered into one or more of the channels and/or between the channels and the outer wall of the overtube. The inflation source can be located at proximal end 106 of overtube 102, and proximal end 106 may include a port for receiving the inflation source. Exemplary inflation sources include a pneumatic source for providing pressurized air or gases and a hydraulic source for providing pressurized fluids. The source for inflating channel 110 may be the same or different than the source for inflating the outer wall of overtube 102.

In one embodiment, channel 110 and overtube 102 are configured such that radially expanding the body of overtube 102 concomitantly expands channel 110 (or conversely expanding channel 110 expands the overtube). For instance, a wall of channel 110 may be attached to inner wall 124 of overtube 102 such that expanding overtube 102 pulls the wall of channel 110 in an outward radial direction.

In an aspect, longitudinal baffles attach at least a portion of overtube 102 to at least a portion of a wall of channel 110. The baffles can transfer force from the overtube to the channel such that expanding the overtube expands the wall of channel 110. The baffles may be made of a substantially non-elastic, but flexible material, including polymers, such as polyimide, polyamide, polytetrafluoroethylene (PTFE), or polyethylene, or, fibers, textiles, and nonwovens. In another aspect, the baffles can be formed of a rigid material such that the spacing between the main channel and outer wall of the overtube is uniform as the overtube expands. Alternatively, the baffles can be formed of elastic, stretchable, and/or deformable materials.

When the overtube is in an insertion configuration (or when the overtube is not fully expanded), the baffles can impart a corrugated appearance to channel 110 where the baffles extend inwardly and fold the wall of channel 110. Regardless, the area between any two adjacent baffles can define channels.

In another aspect, at least one chamber may be used to expand overtube 102. For instance, a chamber configured to expand may be supported by a plurality of spiraling rings. The plurality of spiraling rings may also support overtube 102 in an expanded configuration.

In another aspect, channel 110 and overtube 102 are independently configured to expand, that is, the radial expansion of overtube 102 does not concomitantly radially expand channel 110. Thus, channel 110 may expand at a different rate than overtube 102. The different rate of expansion may be attributed to a different material of construction. In another aspect, the different rate of expansion may be attributed to a different inflation source.

In another aspect, channel 110 radially expands when a medical instrument is passed therethrough. Thus, channel 110 may be configured to accommodate the shape and size of a medical instrument.

The distal end of overtube 102, as mentioned above, can include a distal opening to channel 110. To block the ingress of biological materials during delivery of the overtube, the overtube channels may be covered or blocked by an obturator. Exemplary obturators include piercable membranes and plugs. The obturator can additionally assist with sterility.

Alternatively, overtube 102 can include a one-way valve, for example, a lumen pinched shut during inflation, a flapper valve, a duckbill valve, a check valve, and the like, to prevent backflow of fluids within overtube 102. In another embodiment, channel 110 can include a one-way valve, for example, a lumen pinched shut during inflation, a flapper valve, a duckbill valve, a check valve, and the like, to prevent backflow of fluids within overtube 102.

In one aspect, overtube 102 may include at least one articulation section 112. At least one articulation section 112 can be radially expanded in one embodiment. In one aspect, articulation section 112 provides at least one degree of freedom, and in another aspect, provides more than one degree of freedom (e.g., two, three, or more than three degrees of freedom) to system 100. In one aspect, a user can control side-to-side and/or up/down bending of the articulation section via proximal controls. In another aspect, overtube 102 can additionally, or alternatively, move longitudinally and/or rotate.

The degree to which the articulation section bends can be varied according to the needs of the medical procedure. In one aspect, articulation section 112 can bend up to at least about 180 degrees to allow retroflexing. For example, in a trans-oral approach to a gall bladder or liver, a surgeon may wish to turn in a cranial direction to look toward the diaphragm.

Other procedures may require less bend, such as, for example, a bend of at least about 45 degrees from the longitudinal axis of overtube 102. Exemplary bend angles may include, for instance, at least about 15 degrees, at least about 30 degrees, at least about 45 degrees, at least about 60 degrees, at least about 75 degrees, at least about 90 degrees, at least about 105 degrees, at least about 120 degrees, at least about 135 degrees, at least about 150 degrees, at least about 165 degrees, or at least about 180 degrees, all measured from a longitudinal axis of overtube 102. In addition, or alternatively, overtube 102 can include multiple articulation sections and/or can be adapted to lock in position or increase in stiffness.

In one aspect, where multiple channels are present, only one of the channels is driven via the articulation section. For example, one of the channels can be articulated independently of another channel and/or articulated independently of the overtube body. Alternatively, two or more channels are mated with one another and articulating one channel drives channels mated therewith. Thus, channels 110 can be directly articulated together or independently depending on the intended use of system 20.

A variety of control mechanisms can be used to manipulate articulation section 112, including, for example, push-pull strands, leaf springs, cables, oversheaths, ribbons, electroactive materials, pre-bent material, shape memory material (for example, heat activated materials), and/or fluid actuation. In one embodiment, the control mechanism may be one or more strands 60. Strands 60 may extend from proximal end 106, or the proximal portion, of overtube 102 to articulation section 112 to control the bend of articulation section 112. Strands 60 may comprise one or more filaments formed of a flexible material include, for example, a variety of wires and cables. Strands 60 may include an inner filament positioned within an outer casing.

Strands 60 may be positioned in a variety of locations to bend the articulation section. In one aspect, strands 60 extend along outer wall 126 of overtube 102 to articulation section 112. In another aspect, overtube 102 includes one or more lumens between the outer surface and inner surface sized to receive strands 60. In yet another aspect, strands 60 extend longitudinally along the inner wall of overtube 102.

The number of strands 60 can be chosen based on the desired degrees of freedom for overtube 102. For instance, four strands 60 can extend to articulation section 112 and provide two degrees of freedom for overtube 102. When tensioned, the strands can bend the articulation section 112 by moving a series of articulation segments 62. In one aspect, springs 64 connect the articulation segments 62 and allow the articulation segments to move relative to one another. Strands 60 extend across the articulation section and mate with a distal articulation segment 62 and/or a portion of the overtube distal to the articulation section. When a strand is tensioned, the articulation segments 62 move relative to one another along at least part of articulation section 112 of overtube 102, allowing articulation section 112 to bend.

In another aspect, overtube 102 includes a shape-memory or prebent material that drives movement of the articulation section. Shape-memory alloys are structures that change their shape when exposed to a trigger such as, for example, heat. After exposure to temperatures corresponding to temperatures of biofluids, the articulation section may revert to a curved configuration. The temperatures may be selected to approximate an expected temperature for a body cavity, a body organ, or body system. In one embodiment, the temperature ranges from about 30° C. to about 40° C. In another embodiment, the temperature ranges from about 33° C. to about 38 C. In another embodiment, the temperature ranges from about 35° C. to about 37° C. The articulation section may be constructed to take any useful curved shape, for example, a c-curve, an s-bend, and the like.

Regardless, articulation section 112 can, in one embodiment, radially expand. Articulation section 112 can be constructed of at least one material configured to stretch, flex, and/or deform. The material may be the same or different than the materials forming the other portions of overtube 102.

In one aspect, articulation section 112 includes one or more segments, such as, for example, disclosed in U.S. patent application Ser. No. 11/946,779, entitled DIRECT DRIVE ENDOSCOPY SYSTEMS AND METHODS, which is herein incorporated by reference. In order to permit expansion of channel 110, the segments can radial expand. For example, the segments can be formed of stretchable, deformable, and/or flexible material. However, as used herein, the terms “articulation” and “articulation section” are not limited to structures having a series of interconnected segments.

In another aspect, the articulation segment can include a shaft or tube of flexible, radially expandable material. A lumen or lumens extending through the shaft can define channels 110. Described below are exemplary control mechanisms, for example, pull wires, for driving articulation of the articulation section.

In one aspect, articulation section 112 includes at least one rod formed of at least one shape-memory material or at least one prebent material. The rod can be preshaped to revert to a bent configuration in use. The rod can be positioned along the exterior of overtube 102 in one embodiment. The rod can be mated to overtube 102 by, for example, a mechanical connection, by a suture, or by a band of fiber. In another embodiment, the rod is positioned at a location between outer wall 126 and inner wall 124 of overtube 102. In yet another embodiment, the rod is positioned along the interior of overtube 102. The rod can be mated to overtube 102 by, for example, a mechanical connection, by a suture, or by a band of fiber. In another embodiment, the rod is mated to channel 110.

In an embodiment where overtube 102 is articulated by the use of shape-memory and/or prebent material, overtube 102 may be removed from a patient by inserting an instrument, such as a stiffening rod or rigid instrument, to apply a force to the shape-memory and/or prebent material. The instrument can be configured to apply a pressure to overcome the natural configuration of the movable object formed from the shape-memory and/or elastic material.

Turning now in more detail to the embodiments depicted in the figures, FIG. 1A illustrates overtube 102 according to one aspect. Overtube 102 is generally cylindrical in configuration, and in the aspect depicted in FIG. 1A, has a collapsed, axially aligned channel 110, disposed in about the center of overtube 102. Overtube 102 includes an outer wall 126 and an inner wall 124, one or both of which can be radially expanded. In one aspect, inner wall 124 can be folded while in the overtube is in the insertion configuration. Unfolding of inner wall 124 can permit expansion of channel 110.

Overtube 102 includes a distal end 104 and a proximal end 106. Insertion of overtube 102 in a patient is from distal end 104. Distal end 104 may include visualization and/or illumination devices of the type that are common in endoscopes. Proximal end 106 remains exterior to the patient. Proximal end 106 includes a port 120 where a fluid or gas can be introduced to expand outer wall 126 and/or expand channel 110 to the second configuration. In the insertion configuration, as illustrated in FIG. 1, overtube 102 has a smaller first diameter to facilitate insertion.

In FIG. 1B, channel 110 has also been expanded into a second configuration. As illustrated, channel 110 has a generally circular cross-section along the length of channel 110 and is sized for the passage of one or more medical instruments. In another aspect, channel 110 takes other cross-sectional shapes, for example, elliptical or asymmetric.

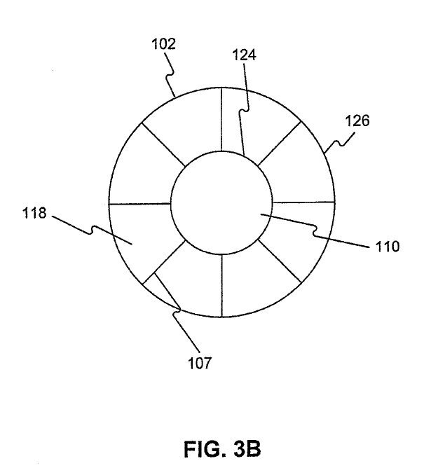

FIG. 3A illustrates a cross-section of overtube 102 including a series of inflation chambers 118 separated by baffles 107. Baffles 107 connect outer wall 124 to inner wall 126 in a radial pattern and generally extend longitudinally within overtube 102 along all or a portion of the length of overtube 102. In addition, baffles 107 can define additional channels within overtube 102. Additionally, or alternatively, baffles 107 can include openings to permit the flow of inflation fluid between chambers 118.

Referring to FIG. 3B, inflation fluid has been delivered into inflation chambers 118, expanding overtube 102. Expanded overtube 102 has a relatively larger second diameter as compared with overtube 102 illustrated in FIG. 3A.

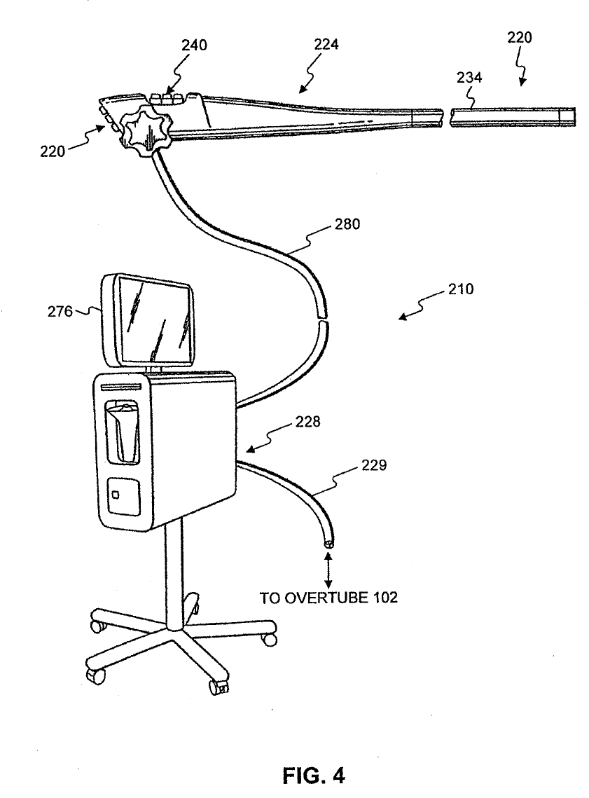

FIG. 4 illustrates one exemplary embodiment of an endoscopic video imaging system 210 that may be used in combination with overtube 102. The system 210 includes an imaging endoscope 220 having a control handle 224, an insertion tube 234, and a communications conduit 280 to connect the endoscope 220 to a control cabinet 228. Overtube 102 may or may not be a part of the endoscopic video imaging system 210. In other words, overtube 102 may optionally receive power and utilities from the system 210. Alternatively, overtube 102 can have a separate control for controlling the visualization/illumination devices and the inflation/deflation of overtube 102. Preferably, to eliminate the need for a separate control and control handle for overtube 102, overtube 102 can be designed to operate with an existing endoscopic system, such as system 210. For example, control cabinet 228 can have auxiliary ports that provide power and utilities through separate terminal connections that are not designated for communications conduit 280. Instead, the auxiliary ports are connected to a second, separate communications conduit 229 designated for overtube 102.

Overtube 102 can include terminal connections for a visualization/illumination cable and port 120 at proximal end 106 that are compatible with the distal, terminal connections on communications conduit 229 (not shown). Communications conduit 229 carries utilities, such as an inflation gas or liquid, to overtube 102 from the existing control cabinet 228.

In an aspect, communications conduit 229 can also serve to functionally interconnect overtube 102 to control cabinet 228 so that overtube 102 is controlled by the one or more switches on control handle 224. Alternatively, a separate control unit other than handle 224 may be used to operate overtube 102. Control cabinet 228 functions to provide image processing capabilities, as well as being capable of supplying power, fluids, air, etc., to endoscope 220 and to overtube 102.

As indicated above, distal end 104 of overtube 102 can include a visualization device of the optical type in which an optical image is carried on a coherent fiber optic bundle, or, alternatively, the video type, in which a miniature image sensor, which includes a charge coupled device (CCD), CMOS imaging sensor, or the like, is powered through electrical cables. Distal end 104 of overtube 102 can include an illumination device, such as LEDs, or light from a light source at the control cabinet carried on a fiber optic bundle. Depending on the type of visualization device and illumination device, overtube 102 may include electrical or fiber optic cables, or both.

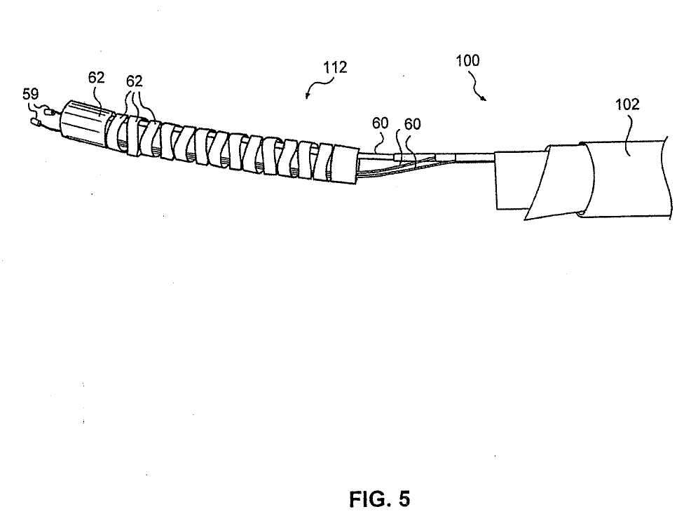

Articulation members of the articulation section of overtube 102 can be controlled by strands. Referring to FIG. 5, segments 62 of articulation section 112 are controlled with strands 60. Strands 60 can be mated with segments 62 and/or with another distal portion of the overtube via, for example, a mechanical connection. In one aspect, segments 62 define a portion of channel 110 and are radially expandable. For example, the segments can be constructed, at least in part, of a flexible, deformable, and/or elastic material that permits radial expansion of a channel positioned therein. Alternatively, or additionally, a radially expandable channel can be mated with articulation segment defined by segments 62. Thus, in one aspect, segments 62 do not radially expand.

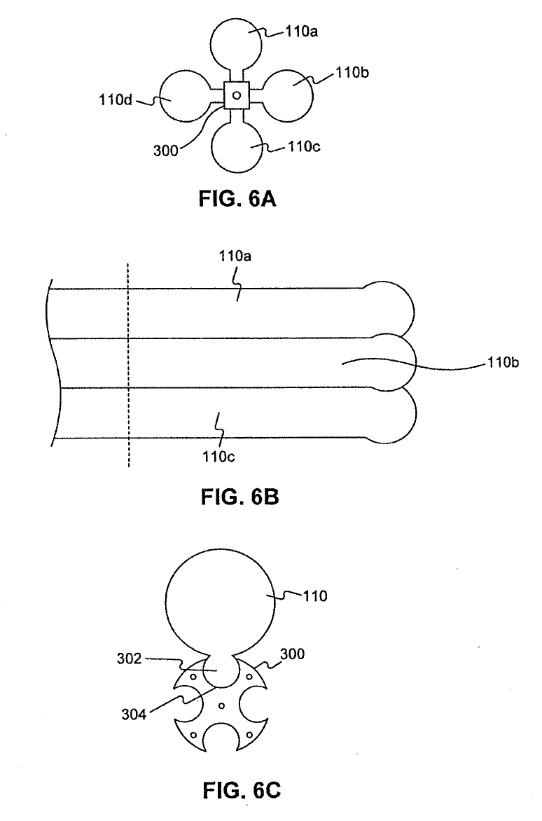

In another embodiment, the articulation section includes an articulation members mated with channel 110. In an embodiment illustrated in FIG. 6A, channels 110a, 110b, 110c, and 110d can mate with a central control shaft. For example, as illustrated in FIGS. 6A and 6B, central control shaft 300 mates with channels 110a, 110b, 110c, and 110d. The channels can surround shaft 300 and/or attach to the outer surface of shaft 300.

Shaft 300 can also include a control mechanism for steering the channels. For example, strands 60 can extend through or along shaft 300 to a distal articulation section. Tensioning the strands can drive one or more degrees of freedom of shaft 300, including, for example, up/down and/or left/right movement.

In one aspect, one or more channels 110a, 110b, 110c, and 110d fixedly mate with shaft 300. In another aspect, the channels and/or movable object are detachably mated with shaft 300.

FIG. 6C illustrates a cross-section of overtube 102 with channel 110 having a surface feature that mates with a surface feature of shaft 300. In the illustrated embodiment, channel 110 includes a mating feature 302 having a curved or c-shaped outer surface corresponding to a mating feature 304 of shaft 300. In use, channel 110 can slide along shaft 300 by slide mating feature 302 within mating feature 304. One skilled in the art will appreciate that a variety of movable mating features could be substituted for mating features 302 and 304.

In another embodiment, channel bodies 110a, 110b, 110c, and 110d can articulate independently of shaft 300 at the distal end of overtube 102. For example, the channels can be detached from shaft 300 and independently moved via, for example, strands and/or shape-memory materials.

Further described herein are methods of using the systems and devices described above. In one embodiment, a radially expandable overtube is provided. The overtube can change from an insertion configuration having an outer diameter that facilitates insertion through an access point and/or body lumen to a larger expanded configuration that facilitates the delivery and/or removal of instruments and/or materials. In one aspect, the outer diameter of the overtube in the insertion configuration is less than the inner diameter of a natural body lumen (e.g., esophagus). Once positioned within the body lumen, the overtube can be expanded to expand the diameter of the body lumen. In particular, the second configuration of the overtube can have an outer diameter that is equal to or greater than the relaxed diameter of the natural body lumen and/or orifice.

In addition to expanding the outer diameter of the overtube, the diameter (or cross-sectional area) of an inner channel can be expanded. In one aspect, the overtube includes a main channel that increases in diameter as the overtube moves between the insertion configuration and the expanded configuration. In another aspect, the overtube can include two or more channels that radially expand. In yet another aspect, all the channels within the overtube radially expand as the overtube moves between the insertion configuration and the second configuration.

One exemplary method includes the steps of inserting the overtube, while in an insertion configuration, through a natural orifice, and then expanding the overtube to accommodate the passage of one or more instruments. In one aspect, expanding the overtube radially expands at least a portion of a natural body lumen.

In one aspect, a method for passing an instrument through an overtube includes inserting an overtube within a body, wherein the overtube is configured to expand in the radial direction and having a main channel configured to expand in the radial direction and at least one additional channel; radially expanding the overtube; radially expanding the main channel; and passing at least one medical instrument through the overtube. For procedures in which the overtube is not inserted through a natural orifice, an incision may first be made at the surgical site, the incision sized to receive the unexpanded overtube.

To expand the overtube, main channel and/or at least one channel, a user may use various techniques. A medical instrument may be passed through the main channel such that the main channel expands to accommodate the size and shape of the medical instrument. If the medical instrument is larger than the unexpanded overtube, passing the medical instrument through the main channel can additionally, or alternatively, drive expansion of the overtube.

The overtube, main channel and/or at least one channel may be expanded by inflation. The proximal end of the overtube can be configured to receive an inflation source. For instance, an inflation source may be mated to a port at the proximal end or a port in an end cap of the proximal end. Exemplary inflation sources may be pneumatic or hydraulic.

The overtube, main channel and/or at least one channel may be expanded by removing a vacuum within the overtube.

The overtube, main channel and/or at least one channel may be expanded by exposing the overtube and/or main channel to biofluids. A property of the biofluids, such as pH, temperature, the chemical structure of the biofluid components, and the like may activate the material of the overtube, main channel and/or at least one channel to expand.

An exemplary class of procedures that the systems described herein can perform includes: cardiovascular; radiology; pulmonary ENT; neurology; orthopedics; gynecology; general surgery; gastrointestinal; and urology.

Also provided are an exemplary list of access points for the systems described herein: trans-oral; trans-anal; trans-vaginal; percutaneous (for example, laparoscopic, thorascopic, to the circulatory system); trans-nasal; and trans-urethral.

While the various embodiments have been illustrated and described, it will be appreciated that various changes can be made therein without departing from the spirit and scope of the invention. It is, therefore, intended that the scope of the invention be determined from the following claims and equivalents thereof.

What is claimed is:

1. A system comprising:
an overtube comprising an elongate body having a distal end and a proximal end,
the overtube configured to radially expand from an insertion configuration to an expanded configuration and including at least one expandable articulation section having at least one degree of freedom;
a main channel configured to expand radially; and
at least one additional channel.

2. The system of claim 1, wherein at least one of the at least one additional channel is configured to expand radially.

3. The system of claim 1, wherein the main channel and the overtube are mated such that radially expanding the overtube concomitantly expands the main channel.

4. The system of claim 3, wherein at least one baffle mates the main channel and the elongate body, the at least one baffle extending at least a portion of the distance between the distal end and the proximal end of the overtube.

5. The system of claim 1, wherein the proximal end comprises a port configured to receive an inflation source for expanding the overtube.

6. The system of claim 1, wherein the overtube is flexible.

7. The system of claim 1, wherein a wall defining the main channel comprises a flexible, deformable, and/or stretchable material the permits radial expansion of the main channel.

8. The system of claim 7, wherein the material is at least one of an elastic, a polymeric, or an elastomeric material.

9. The system of claim 1, wherein the overtube is non-self-collapsible.

10. The system of claim 9, comprising a source of vacuum pressure, wherein the overtube is in the insertion configuration when under vacuum.

11. The system of claim 10, comprising a cell foam material configured to collapse when under vacuum.

12. The system of claim 1, wherein radially expanding the overtube radially expands the main channel and at least one of the at least one channel.

13. The system of claim 1, further comprising an instrument having a diameter greater than an inner diameter of the main channel in the insertion configuration.

14. The system of claim 13, wherein the instrument is an endoscope.

15. The system of claim 1, wherein the articulation section is configured to bend up to at least 180 degrees.

16. The system of claim 1, wherein at least one of the at least one channel is configured to articulate separately from the overtube.

17. The system of claim 1, wherein the overtube is fluid tight.

18. The system of claim 1, further comprising an optical device fixedly mated proximate to the distal end of the overtube.

19. The system of claim 1, further comprising an illumination device proximate to the distal end of the overtube.

20. A system comprising:
an overtube comprising an elongate body having a distal end and a proximal end,
the overtube configured to radially expand from an insertion configuration to an expanded configuration and having at least one expandable articulation section having at least one degree of freedom and having an expandable optical channel configured to receive an optical device.

21. The system of claim 20, wherein the optical device is an endoscope.

22. The system of claim 20, wherein all the channels within the overtube are radially expandable.

23. A method for passing an instrument through an overtube, comprising:
inserting an overtube within a body, wherein the overtube is configured to expand in the radial direction and having a main channel configured to expand in the radial direction and at least one additional channel;
radially expanding the overtube;
radially expanding the main channel; and
passing at least one medical instrument through the overtube.

24. The method of claim 23, wherein the step of radially expanding the overtube concomitantly radially expands the main channel.

25. The method of claim 23, comprising an initial step of making an incision at a surgical site.

26. The method of claim 23, wherein the step of passing the medical instrument radially expands the main channel.

27. The method of claim 23, wherein the medical instrument is an endoscope.

28. The method of claim 23, wherein the medical instrument is a surgical instrument.

29. The method of claim 23, wherein the medical instrument is a diagnostic instrument.

30. The method of claim 23, wherein the overtube comprises at least one articulation section having at least two degrees of freedom.

31. The method of claim 23, wherein the step of expanding the overtube comprises inflating the overtube.

32. The method of claim 23, wherein the overtube comprises a cell foam material configured to collapse when under vacuum, and the step of expanding the overtube comprises removing the vacuum source.

33. The method of claim 23, comprising passing a fluid through at least one of the main channel or the at least one additional channel.

34. The method of claim 23, comprising radially expanding at least one of the at least one additional channel.

35. The method of claim 23, wherein expanding the main channel comprises inflating the main channel.

36. The method of claim 23, comprising the step of passing a diagnostic instrument and then inserting a surgical instrument.

37. The method of claim 36, wherein the diagnostic instrument is removed prior to the insertion of the surgical instrument.

38. The method of claim 36, wherein the step of inserting the surgical device comprises expanding the overtube to accommodate a surgical instrument.
